(19) United States
(12) Patent Application Publication
(10) Pub. No.: US 2011/0046389 A1
(43) Pub. Date: Feb. 24, 2011

(54) INTERMEDIATES FOR THE PREPARATION OF (3R, 4S)-1-(4-FLUOROPHENYL)-3-[(3S)-3-(4-FLUOROPHENYL)-3-HYDROXYPROPYL)]-4-(4-HYDROXYPHENYL)-2-AZETIDINONE

(76) Inventors: Hana Stepankova, Cesky Brod (CZ); Josef Hajicek, Praha (CZ); Marketa Slavikova, Libcice nad Vltavou (CZ); Josef Zezula, Polna (CZ)

Correspondence Address: Pearl Cohen Zedek Latzer, LLP, 1500 Broadway, 12th Floor, New York, NY, 10036, US

(21) Appl. No.: 12/918,869

(22) PCT Filed: Feb. 13, 2009
(86) PCT No.: PCT/CZ2009/000016

§ 371 Date: Oct. 28, 2010

(30) Foreign Application Priority Data
Feb. 25, 2008 (CZ) PV 2008 107

(51) Int. Cl. C07D 263/20

(52) U.S. Cl. 548/230

(57) ABSTRACT

A method for the preparation of (S)-alcohol oxazolidides of general formula II, in which PG represents hydrogen or a hydroxyl protecting group, such as trimethylsilyl, tert-butyldimethylsilyl, benzyloxycarbonyl, tert-butoxycarbonyl, benzyl, benzhydryl or trityl, in which a ketal oxazolidide of general formula III, where PG has the same meaning as above and R means an alkyl with 1-4 carbon atoms, linear or branched, such as methyl, ethyl, isopropyl or butyl, or R+R together represents a divalent alkyl, or substituted with 1 or 2 alkyl groups, e.g. 1,2-ethylene, 1,2-propylene, 1,2-butylene, 1,3-propylene or 2,2-dimethyl-1,3-propylene, is deprotected by the action of acidic reagents in a mixture of water and a water-miscible solvent in the temperature range of 0 to 100° C. (stage A), and the obtained ketone oxazolidide of general IV, in which PG has the same meaning as above, is reduced with asymmetrical reagents in an inert organic solvent in the temperature range of −30 to +40° C. (stage B).

### TECHNICAL FIELD

The invention deals with a new method for the preparation of O-protected (4S)-3-{(2R,5S)-5-(4-fluorophenyl)-2-[(S)-[(4-fluorophenyl)amino](4-hydroxyphenyl)methyl]-5-hydroxypentanoyl}-4-phenyl-1,3-oxazolidin-2-ones.

### BACKGROUND ART

(3R,4S)-1-(4-fluorophenyl)-3-[(3S)-3-(4-fluorophenyl)-3-hydroxypropyl)]-4-(4-hydroxyphenyl)-2-azetidinone of formula (I),

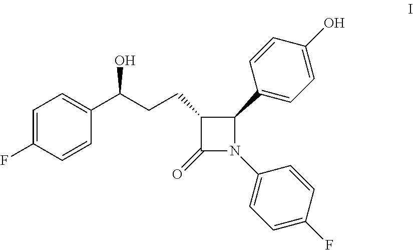

known under the INN name ezetimibe, is described in U.S. Pat. No. 5,631,365 as a hypolipidemic agent reducing intestinal absorption of cholesterol and other sterols.

According to U.S. Pat. Nos. 5,739,321 and 5,886,171 ezetimibe is produced in such a way that (S)-4-hydroxybutanolide is added onto N-(4-benzyloxybenzylidene)-4-fluoroaniline with the use of LDA at −78° C., the obtained diol is split with a periodate to an aldehyde, which reacts with 4-fluoroacetophenone O-trimethylsilylenole producing an aldol. The aldol is dehydrated to produce an unsaturated ketone whose double bond, or also the benzyl protecting group at the same time, are hydrogenated on a palladium catalyst. Then the ketone is asymmetrically reduced with a borane in the presence of a chiral ligand to produce ezetimibe, or its O-benzyl derivative, which is hydrogenolyzed on a palladium catalyst. A disadvantage of this method consists in the necessity to work at very low temperatures and in the repeated use of expensive catalysts of the palladium type.

The production method of ezetimibe described in U.S. Pat. No. 5,856,473 starts from 5-(4-fluorophenyl)-4-pentenoic acid, which is converted to a chloride with the use of oxalyl chloride and further on, by reaction with (S)-4-phenyl-2-oxazolidinone, to acyl oxazolidide. The latter is added onto N-(4-benzyloxybenzylidene)-4-fluoroaniline with the use of titanium tetrachloride in the presence of diisopropylethylamine to provide a product, which is cyclized using bistrimethylsilylacetamide and catalytic TBAF to produce olefine azetidinone. This alkene is converted to a ketone by the action of Pd(OAc)₂ and benzoquinone in the presence of perchloric acid. The ketone is again asymmetrically reduced with a borane in the presence of a chiral ligand and finally hydrogenolysis of the O-benzyl protecting group is performed. A considerable disadvantage of this method is the repeated use of expensive catalysts of the palladium type again and the use of toxic oxalyl chloride.

According to the above mentioned U.S. Pat. No. 5,631,365 ezetimibe is produced in such a way that (S)—N-(4-methoxycarbonylbutanoyl)oxazolidide is synthesized from (S)-4-phenyl-2-oxazolidinone and glutaric acid ester chloride and then it is added in the presence of titanium tetrachloride onto the above mentioned N-(4-benzyloxybenzylidene)-4-fluoroaniline and the obtained product is cyclized by the action of bistrimethyl silyl acetamide and catalytic TBAF to give an ester azetidinone Alkaline hydrolysis of the ester results in an acid, which is converted to an acid chloride, whose reaction with a Grignard reagent in the presence of ZnCl₂ and Pd(PPh₃)₄ produces a ketone. The latter is asymmetrically reduced with a diborane in the presence of a chiral ligand and finally hydrogenolysis of the O-benzyl protecting group is performed on a palladium catalyst. Also in this case a considerable disadvantage consists in the use of expensive catalysts of the palladium type as well as the use of toxic oxalyl chloride.

The production method of ezetimibe in accordance with WO 2006/137080 is similar to the above mentioned one and it also has similar disadvantages. Methyl ester chloride of glutaric acid is produced by the action of oxalyl chloride on the corresponding acid and is reacted with (S)-4-phenyl-2-oxazolidinone to produce (S)—N-(4-methoxycarbonylbutanoyl)-oxazolidide. The latter is added in the presence of titanium tetrachloride onto the above mentioned N-(4-benzyloxybenzylidene)-4-fluoroaniline, and the obtained product is cyclized by the action of bistrimethylsilylacetamide and catalytic TBAF to an ester-azetidinone. Alkaline hydrolysis of the ester provides an acid, which is converted with the use of oxalyl chloride to the acyl chloride, whose reaction with a Grignard reagent in the presence of ZnCl₂ and acetate of a transitional metal, such as palladium, produces a ketone. The ketone is asymmetrically reduced with a diborane in the presence of a chiral ligand and finally hydrogenolysis of the O-benzyl protective group is performed on a palladium catalyst. This method also manifests the considerable disadvantage of the repeated use of expensive catalysts of the palladium type as well as repeated use of toxic oxalyl chloride.

The production method of ezetimibe in accordance with WO 2007/072088 starts from 4-(4-fluorobenzoyl)butanoic acid, which is first converted to ethylene ketal and then, by reaction with (S)-4-phenyl-2-oxazolidinone, to (S)-3-[4-[2-(4-fluorophenyl)-[1,3]-dioxolan-2-yl]butanoyl]-4-phenyl oxazolidin-2-one. Its addition to O-silylated N-(4-hydroxybenzylidene)-4-fluoroaniline by the action of titanium isopropoxide trichloride provided a product, which was cyclized with the use of bistrimethylsilylacetamide and catalytic TBAF to ketal azetidinone and deprotected to ketone azetidinone with the use of montmorillonite K10. The silylated ketone produced this way was reduced with diborane in the presence of chiral (R)-o-tolyl-CBS-oxazaborolidine. The obtained O-silylated ezetimibe with de >98% was finally deprotected with sulfuric acid in isopropyl alcohol.

The production method in accordance with WO 2007/119106 comprises not only the above mentioned ketal, (S)-3-[4-[2-(4-fluorophenyl)-[1,3]-dioxolan-2-yl]butanoyl]-4-phenyl oxazolidin-2-one, but also its analog derived from 1,3-propanediol. Their addition onto O-benzylated or trimethylsilylated N-(4-hydroxybenzylidene)-4-fluoroaniline by the action of titanium isopropoxide trichloride provided products, which were cyclized to ketal azetidinones with the use of bistrimethylsilylacetamide and catalytic TBAF and then deprotected to ketone azetidinones with the use of p-toluene sulfonic acid in acetone. The benzyloxy ketone produced this way was reduced with a borane in the presence of chiral (R)-2-methyl-CBS-oxazaborolidine and subsequently deprotected by hydrogenation on Pd/C. Ezetimibe was alternatively obtained with the use of the same CBS reduction of the hydroxy ketone.

A similar method of synthesis of ezetimibe from 5- and 6-membered ketals and protected imines is also described in patent application No. WO 2007/120824.

A common problem of these three methods is chemoselectivity and diastereoselectivity of the CBS reduction of ketones with a borane and subsequent laborious final purification of the produced ezetimibe.

### DISCLOSURE OF INVENTION

The invention deals with a method for the preparation of (S)-alcohol-oxazolidides of general formula II

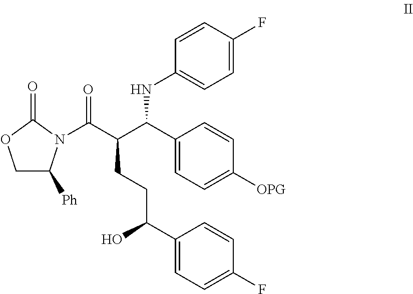

wherein PG represents hydrogen or a hydroxyl protecting group, such as trimethylsilyl, tert-butyldimethylsilyl, benzyloxycarbonyl, tert-butoxycarbonyl, benzyl, benzhydryl or trityl, the essence of which is that ketal oxazolidide of general formula III

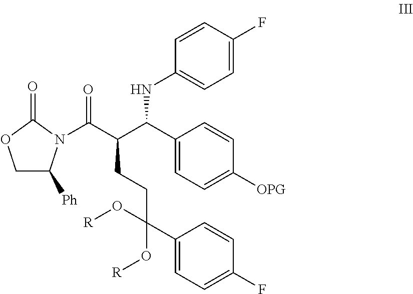

wherein PG means the same as above and R represents an alkyl with 1-4 carbon atoms, linear or branched, such as methyl, ethyl, isopropyl or butyl, or R+R together represent a divalent alkyl, optionally substituted with 1 or 2 alkyl groups, e.g. 1,2-ethylene, 1,2-propylene, 1,2-butylene, 1,3-propylene or 2,2-dimethyl-1,3-propylene,
is deprotected by the action of acidic reagents in a mixture of water and a water-miscible solvent in the temperature range of 0 to 100° C. (stage A),
and the obtained ketone oxazolidide of general formula IV

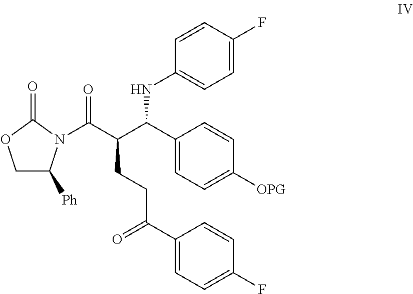

wherein PG has the same meaning as above,
is reduced with asymmetrical reagents in an inert organic solvent in the temperature range of −30 to +40° C. (stage B).

We have found out that O-protected (4S)-3-{(2R)-5-(4-fluorophenyl)-2-[(S)-[(4-fluorophenyl)amino](4-hydroxyphenyl)methyl]-5-oxopentanoyl}-4-phenyl-1,3-oxazolidin-2-ones (ketone oxazolidides hereinafter) of general formula IV and O-protected (4S)-3-{(2R,5S)-5-(4-fluorophenyl)-2-[(S)-[(4-fluorophenyl)amino](4-hydroxyphenyl)methyl]-5-hydroxypentanoyl}-4-phenyl-1,3-oxazolidin-2-ones ((S)-alcohol oxazolidides hereinafter) of general formula II, wherein PG has the above mentioned meaning, are novel and represent important intermediates in industrial, well-realizable production of ezetimibe of formula I. Those compounds of general formulas II and IV have proved to be preferable in which PG represents the trimethylsilyl, tert-butyldimethylsilyl, benzyloxycarbonyl, tert-butoxycarbonyl, benzyl, benzhydryl or trityl groups, and out of them the particularly preferred benzyloxycarbonyl, tert-butyldimethylsilyl and benzyl groups.

We have also found out that the reduction of ketones of general formula IV to (S)-alcohols of general formula II with the use of the CBS borane method is not only highly diastereoselective, but also highly chemoselective. The high chemoselectivity is enabled by the fact that the sensitive azetidinone group is not present in the molecules of general formulas II and IV. This fact contributes to the advantageousness of the method of production of ezetimibe of formula I from protected ketone oxazolidides of general formula III via ketone oxazolidides of general formula IV and (3S)-alcohol oxazolidides of general formula II.

Stage A. The ketal of general formula III, wherein PG and R have the same meaning as above, is hydrolyzed by the action of acidic reagents such as organic acids, e.g. p-toluenesulfonic acid, methanesulfonic acid, acetic acid, or inorganic acids, e.g. hydrochloric acid, in a mixture of water and a water-miscible solvent, such as tetrahydrofuran, acetone, methyl ethyl ketone or isobutyl methyl ketone, or an alcohol, e.g. methanol or ethanol, in the temperature range of 20 to 100° C., preferably at 50° C. up to the boiling temperature of the mixture.

Stage B. The ketones of general formula IV, in which PG and R have the same meaning as above, are reduced with asymmetrical reagents in an inert organic solvent in the temperature range of −30 to +40° C. As the asymmetrical reagent a borane is used in the presence of a chiral ligand or a hydrogen source in the presence of a chiral catalyst.

If a borane in the presence of a chiral ligand is used, the borane source can be a borane complex, for example with dimethyl sulfide, tetrahydrofuran, dimethyl aniline or diethyl aniline, and a 2-substituted (R)-CBS-oxazaborolidine can be used as the chiral ligand, such as (R)-2-methyl-CBS-oxazaborolidine or (R)-2-(o-tolyl)-CBS-oxazaborolidine in an amount of 1 to 100 mol %, preferably 5 to 25 mol %. The reduction is carried out in the presence of a catalytic amount of a protic or Lewis acid, such as methanesulfonic acid, p-toluenesulfonic acid, trifluoroacetic acid, borotrifluoride etherate or β-chlorodiisopinocamphenyl borane.

Suitable inert organic solvents are e.g. tetrahydrofuran, 2-methyltetrahydrofuran, tert-butylmethylether, toluene or dichloromethane or their mixtures. The reduction is preferably carried out at the temperatures of −25 to −15° C., or at 20 to +30° C.

If an asymmetrical reagent consisting of a source of hydrogen in the presence of a chiral catalyst is used, it is the case of an asymmetrical homogenous reduction. As the source of hydrogen either hydrogen itself or its source such as formic acid or its salts, e.g. triethyl ammonium formate, or isopropyl alcohol can be used. As the chiral catalyst a complex of a transitional metal is used, e.g. of iron, rhodium and ruthenium and their combinations, in the presence of a chiral ligand, or a complex of the above mentioned transitional metals with a chiral ligand embedded in the molecule, preferably e.g. (R)-4-isopropyl-2-[(R)-2-(diphenylphosphino)ferrocen-1yl]oxazoline triphenylphosphino ruthenium(II) chloride. It is advantageous to prepare chiral catalysts in situ.

This invention also comprises a new method for the preparation of O-protected (4S)-3-{(2R)-5-(4-fluorophenyl)-2-[(S)-[(4-fluorophenyl)amino](4-hydroxyphenyl)methyl]-5-oxopentanoyl}-4-phenyl-1,3-oxazolidin-2-ones (hereinafter ketone oxazolidides) of general formula IV

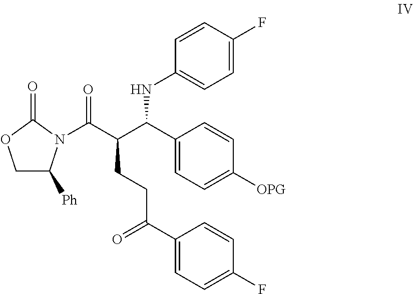

in which PG represents hydrogen or a hydroxyl protecting group such as trimethylsilyl, tert-butyldimethylsilyl, benzyloxycarbonyl, tert-butoxycarbonyl, benzyl, benzhydryl or trityl, starting from (S)-3-[5-(4-fluorophenyl)-1,5-oxopentyl]-4-phenyloxazolidin-2-one of formula V

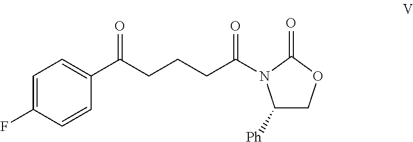

which is ketalized by reaction with a monohydric alcohol of general formula VI

R—OH VI

wherein R represents an alkyl with 1-4 carbon atoms, linear or branched, such as methyl, ethyl, isopropyl or butyl,
in the presence of an accelerator in the temperature range of 10 to 100° C. (stage 1), the resulting ketal oxazolidide of general formula VII

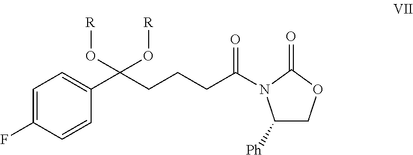

in which R means the same as above,
is reacted with a protected imine of general formula VIII

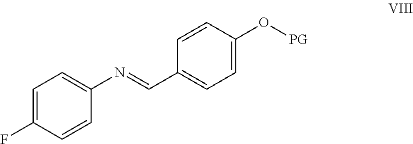

wherein PG has the same meaning as above,
in the presence of a Lewis acid and a strong organic base in an inert organic solvent in the temperature range of −40 to 0° C. (stage 2),
and the resulting ketal oxazolidide of general formula III

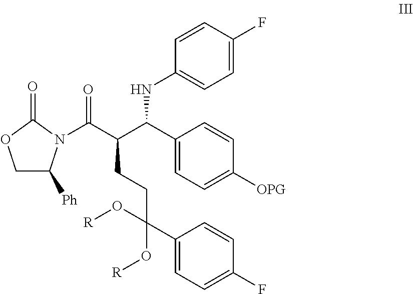

wherein R as well as PG have the meaning mentioned above,
is deprotected by the action of acidic reagents in a mixture of water and a water-miscible solvent in the temperature range of 0 to 100° C. (stage 3).

We have found out that the ketone oxazolidides of general formula IV, in which PG represents hydrogen or a hydroxyl protecting group, such as trimethylsilyl, tert-butyldimethylsilyl, benzyloxycarbonyl, tert-butoxycarbonyl, benzyl, benzhydryl or trityl, can be preferably produced by a method that uses protection of the carbonyl in the compound of formula V in the form of dialkylacetals of general formula VII. A great advantage of the method consists in the fact that the acetal oxazolidides of formula III, obtained by reaction with the imines of general formula VIII are very easily acidically deprotected to produce the desired ketones of general formula IV.

This production method of ketone oxazolidides of formula IV consists in three stages, which are described in detail below.

Stage 1. (S)-3-[5-(4-fluorophenyl)-1,5-oxopentyl]-4-phenyloxazolidin-2-one of formula V is ketalized by reaction with R—OH alcohols of general formula VI, in which R means an alkyl with 1-4 carbon atoms, linear or branched, such as methyl, ethyl, isopropyl or butyl, in the presence of an accelerator in the temperature range of 10 to 100° C., preferably from 50° C. to the boiling temperature of the mixture. As the accelerator a strong mineral or organic acid is used, such as sulfuric acid or p-toluenesulfonic acid in the presence of a water-withdrawing agent, such as a molecular sieve, preferably trialkyl orthoformate such as trimethyl orthoformate or triethyl orthoformate. Preferably, trimethyl orthoformate is used with higher-boiling alcohols R—OH and the resulting methanol is separated by rectification.

Stage 2. (S)-ketal oxazolidides of general formula VII, in which R has the meaning mentioned above, is subjected to a reaction with protected imines of general formula VIII, wherein PG is hydrogen or a hydroxyl protecting group, such as trimethylsilyl, tert-butyldimethylsilyl, benzyloxycarbonyl, tert-butoxycarbonyl, benzyl, benzhydryl or trityl, in the presence of a Lewis acid, e.g. titanium tetrachloride or titanium trichloride alkoxide, in an amount of 1 to 2 equivalents, preferably 1.1 to 1.4 equivalents. The addition is carried out in the presence of a strong organic base, preferably diisopropylethylamine, in an amount of 2 to 5 equivalents, in an inert organic solvent such as dichloromethane, dichloroethane, toluene, tert-butylmethylether, tetrahydrofuran, 2-methyltetrahydrofuran, in the temperature range of −40 to 0° C., preferably at −35 to −15° C.

The procedure can be advantageously carried out by the one-pot method in such a way that first a protected imine of general formula VIII is prepared in situ by reaction of imine VIII (PG=H) with protecting agents PG-X, in which PG has the above mentioned meaning and X is a leaving group, such as chlorine, and then acetal oxazolidide of formula VII is added onto it in the above mentioned manner.

Stage 3. The ketal of general formula III, in which PG and R have the same meaning as above, is hydrolyzed by the action of acidic reagents, such as organic acids, e.g. p-toluenesulfonic acid, methanesulfonic acid, acetic acid, or inorganic acids, e.g. hydrochloric acid, in a mixture of water and a water-miscible solvent, such as tetrahydrofuran, acetone, methyl ethyl ketone, or isobutyl methyl ketone, or an alcohol, e.g. methanol or ethanol, in the temperature range of 10 to 100° C., preferably from 20° C. to the boiling temperature of the mixture.

The following examples illustrate the generic method of the invention but do not limit it in any way.

### EXAMPLE 1

### Preparation of (S)-3-[5,5-dimethoxy-5-(4-fluorophenyl)-1-oxopentyl]-4-phenyloxazolidin-2-one

To a suspension of 15.0 g (42.15 mmol) of (S)-3-[4-(4-fluorobenzoyl)-1-oxobutyl]-4-phenyloxazolidin-2-one in methanol (300 ml) trimethyl orthoformate (22.2 g, 0.21 mol, 5 equiv.) and p-toluenesulfonic acid (0.25 g) are added. The mixture is stirred at the laboratory temperature for 18 h and the obtained solution is then heated at the boiling temperature for 5 h. After cooling, toluene (400 ml) and a 9% solution of NaHCO₃ (170 ml) are added. The separated aqueous phase is extracted with toluene (100 ml). The combined organic phases are washed with water (150 ml) and evaporated after drying.

Yield: 17.5 g of a vitreous substance. HPLC: purity 95.5%.

¹H-NMR (250 MHz, CDCl₃): δ 7.44-7.19 (m, 7H), 6.99 (t, J=8.8 Hz, 2H), 5.33 (dd, J=8.7 Hz, J=3.6 Hz, 1H), 4.59 (t, J=8.8 Hz, 1H), 4.19 (dd, J=8.9 Hz, J=3.6 Hz, 1H), 3.10 (s, 3H), 3.08 (s, 3H), 2.79 (t, J=7.4 Hz, 2H), 1.93-1.82 (m, 2H), 1.36-1.20 (m, 2H).

### Preparation of the Compound of General Formula IV (PG=Cbz)

To a suspension of N-(4-hydroxybenzylidene)-4-fluoroaniline (3.73 g; 17.35 mmol) in dichloromethane (46 ml) diisopropylethylamine (9.5 ml, 55.5 mmol) is added under stirring and cooling to 5° C. and then a 50% toluene solution of benzyl chloroformate (6.5 ml, 19.08 mmol; 1.1 equiv.) is added during 5 minutes. The obtained solution is left to heat up to 10° C. during 1 hour, the course of the reaction being monitored by means of TLC. Then, the solution is cooled down to −30° C., and under stirring a solution of (S)-3-[5,5-dimethoxy-5-(4-fluorophenyl)-1-oxopentyl]-4-phenyl oxazolidin-2-one (5.58 g, corresponding to 13.44 mmol) in dichloromethane (25 ml) is added during 5 minutes. The mixture is further cooled down to −33° C., and after 45 min of stirring, a solution of TiCl₃(Oi-Pr) starts to be added during 30 minutes which has been prepared in advance by mixing of TiCl₄ (1M solution in CH₂Cl₂; 13.0 ml, 13.0 mmol) and Ti(Oi-Pr)₄ (1.32 ml, 4.4 mmol) in CH₂Cl₂ (21 ml) at 10° C., followed by stirring for 45 min. The resulting dark solution is stirred at the same temperature for 3 h and then acetic acid (3.6 ml) is added during 5 minutes and the stirring is continued at −33° C. for 5 min. Then, a 0.46M solution of sodium dihydrogen citrate (90 ml) is added to the reaction mixture. The reaction mixture is intensively stirred for 30 min, the organic phase is separated and the aqueous phase is extracted with CH₂Cl₂ (40 ml). The combined organic phases are washed with water (40 ml), dried (Na₂SO₄) and evaporated in a rotational vacuum evaporator. To the evaporation residue (13.3 g) methanol (70 ml) is added and the suspension is heated up to the boiling point for 1 h and then maintained at the laboratory temperature for 2 h. The separated product is filtered off and washed with MeOH (2×20 ml) and dried at 45° C. Melting temp. 178-179° C.

Yield: 5.55 g (58.6%) of the compound of formula IV (PG=Cbz). HPLC: diastereoisomeric purity 98.7%.

¹H-NMR (250 MHz, CDCl₃): δ 7.87 (m, 2H), 7.50-7.34 (m, 6H), 7.27-6.99 (m, 10H), 6.74 (t, J=8.7 Hz, 2H), 6.37 dd, J=9.0 Hz, J=4.4 Hz, 2H), 5.42 (dd, J=8.5 Hz, J=3.5 Hz, 1H), 5.26 (s, 2H), 5.03 (d, J=9.8 Hz, 1H), 4.65 (t, J=8.6 Hz, 1H), 4.59 (dt, J=8.5 Hz, J=4.6 Hz, 1H), 4.47 (m, 1H), 4.18 (dd, J=8.8 Hz, J=3.5 Hz, 1H), 2.88 (dt, J=7.3 Hz, J=1.4 Hz, 2H), 2.21 (m, 1H), 1.85 (m, 1H).

### Preparation of the Compound of Formula II (PG=Cbz)

To a solution of a ketone of general formula IV (PG=Cbz) (6.00 g, 8.51 mmol) in dry THF (200 ml) a 1M solution of the (R)-Me-CBS catalyst (2.12 ml, 2.12 mmol, 25 mol %) is added at the laboratory temperature under argon. The reaction mixture is stirred for 10 min and then a 1M solution of BH₃.Me₂S in CH₂Cl₂ (11.9 ml, 11.9 mmol, 1.4 equiv.) is added dropwise during 1 hour. After stirring for another 40 min the reaction is terminated by slow addition of methanol (20 ml) under cooling to 10° C. after a TLC check. Then, a 1M aqueous HCl (50) and dichloromethane (100 ml) are added and after stirring for 10 min the organic fraction is separated, washed with water (50 ml) and dried with sodium sulfate. Filtration and evaporation provides a crude product as a solid foam, which is boiled with methanol (120 ml) and then crystallized overnight. The precipitated crystals are sucked off, washed with methanol (15 ml) and dried.

Yield: 5.62 g, i.e. 93.4% of the alcohol of general formula II (R=Cbz). Melting temp. 165-167° C., HPLC: diastereoisomeric purity 98.6%.

¹H-NMR (250 MHz, CDCl₃): δ 7.47-7.35 (m, 5H), 7.27-7.03 (m, 11H), 6.99 (t, J=8.7 Hz, 2H), 6.73 (t, J=8.7 Hz, 2H), 6.34 (m, 2H), 5.39 (dd, J=8.5 Hz, J=3.3 Hz, 1H), 5.26 (s, 2H), 4.97 (d, J=10.1 Hz, 1H), 4.65 (t, J=8.7 Hz, 1H), 4.60-4.49 (m, 2H), 4.37 (dd, J=10.1 Hz, J=8.4 Hz, 1H), 4.18 (dd, J=8.8 Hz, J=3.4 Hz, 1H), 1.86 (d, J=3.4 Hz, 1H), 1.85-1.41 (m, 4H).

¹H-NMR (250 MHz, CD₃SOCD₃): δ 7.50-7.36 (m, 7H), 7.30 (m, 2H), 7.25-7.07 (m, 7H), 7.06 (t, J=8.7 Hz, 2H), 6.79 (t, J=8.8 Hz, 2H), 6.55 (dd, J=9.1 Hz, J=4.5 Hz, 2H), 6.10 (d, J=9.7 Hz, 1H), 5.55 (dd, J=8.5 Hz, J=4.5 Hz, 1H), 5.27 (s, 2H), 4.75 (t, J=8.7 Hz, 1H), 4.53-4.27 (m, 3H), 4.08 (dd, J=8.7 Hz, J=4.6 Hz, 1H), 1.47 (m, 2H), 1.29 (m, 2H).

### EXAMPLE 2

### Preparation of the Compound of General Formula IV (PG=Bn)

A suspension of the ketal of general formula III (R+R=CH₂CH₂, PG=Bn) (8.5 g, 12.06 mmol) and p-toluenesulfonic acid (0.5 g) in a mixture of acetone (320 ml) and water (35 ml) is heated up to boiling while being stirred. After completion of the reaction at this temperature (TLC, 4 h) the reaction mixture is concentrated to ca. ½ volume and crystallized at the laboratory temperature. The crystals are sucked off, washed with cold acetone (15 ml), and dried at 50° C. Melting temp. 176.5-178° C.

Yield: 7.59 g, i.e. 95.3% of the ketone of general formula IV (PG=Bn). HPLC: diastereoisomeric purity 98.8%.

¹H-NMR (250 MHz, CDCl₃): δ 7.86 (m, 2H), 7.49-7.25 (m, 6H), 7.21-6.99 (m, 8H), 6.85 (d se str., J=8.8 Hz, 2H), 6.74 (t, J=8.8 Hz, 2H), 6.39 dd, J=9.0 Hz, J=4.5 Hz, 211), 5.45 (dd, J=8.5 Hz, J=3.3 Hz, 1H), 4.99 (s, 2H), 4.65 (t, J=8.6 Hz, 1H), 4.55 (dt, J=8.8 Hz, J=4.5 Hz, 1H), 4.40 (m, 1H), 4.18 (dd, J=8.8 Hz, J=3.4 Hz, 1H), 2.88 (t, J=7.3 Hz, 2H), 2.17 (m, 1H), 1.80 (m, 1H).

### EXAMPLE 3

### Preparation of the Compound of Formula II (PG=Bn)

2.00 g (3.03 mmol) of the compound of general formula IV (PG=Bn) are dissolved in 100 ml of dry THF in an inert atmosphere. At the laboratory temperature and under stirring a 1M solution of (R)-2-methyl-CBS-oxazaborolidine in toluene (0.75 ml, 0.25 equiv.) is added to this solution. The mixture is stirred for 10 min and then a1M solution of BH₃.Me₂S in dichloromethane (4.24 ml) is added dropwise at the room temperature within 1 h. After the addition is complete the reaction mixture is stirred for another 30 minutes (TLC), then carefully decomposed with methanol (7 ml) and after stirring for 30 min it is diluted with a 1M HCl solution (25 ml). The mixture is extracted with dichloromethane (100 ml) and the combined organic fractions are washed with water (40 ml) and dried with anhydrous sodium sulfate. The organic solvents are evaporated in a vacuum evaporator and the crude product is boiled with methanol (80 ml) and then crystallized at the laboratory temperature for 3 h. The separated crystals are sucked off, washed with methanol (10 ml) and dried at 50° C.

Yield: 1.57 g (78.5%) of an alcohol of general formula II (PG=Bn), melting temp. 172.5-174° C. HPLC: diastereoisomeric purity 98.7%.

¹H-NMR (250 MHz, CDCl₃): δ 7.44-7.28 (m, 5H), 7.22-7.01 (m, 9H), 6.96 (t, J=8.8 Hz, 2H), 6.83 (d, J=8.6 Hz, 2H), 6.71 (t, J=8.6 Hz, 2H), 6.35 (m, 2H), 5.39 (dd, J=8.4 Hz, J=3.3 Hz, 1H), 5.00 (s, 2H), 4.84 (d, J=9.8 Hz, 1H), 4.61 (t, J=8.7 Hz, 1H), 4.58-4.45 (m, 2H), 4.30 (t, J=9.1 Hz, 1H), 4.16 (dd, J=8.8 Hz, J=3.3 Hz, 1H), 1.86 (d, J=3.5 Hz, 1H), 1.81-1.54 (m, 3H), 1.43 (m, 1H).

### EXAMPLE 4

### Preparation of the Compound of Formula IV (PG=Cbz)

A suspension of the ketal of general formula III (R+R=CH₂CH₂CH₂, PG=Cbz) (3.83 g, 5.02 mmol) and p-toluenesulfonic acid (0.21 g) in a mixture of acetone (120 ml) and water (22 ml) is heated up to boiling while being stirred and is maintained at this temperature for 3 h (TLC). The reaction mixture is concentrated in a rotational vacuum evaporator to ca. ½ volume and crystallized at the laboratory temperature. The crystals are sucked off, washed with cold acetone (5 ml), and dried at 40° C. Melting temp. 178.5-179.5° C.

Yield: 3.44 g, i.e. 97.2% of the ketone of general formula IV (R=Cbz). HPLC: purity 98.2%.

### EXAMPLE 5

To a suspension of N-(4-hydroxybenzylidene)-4-fluoroaniline (4.30 g; 20.0 mmol) and trityl chloride (5.91 g, 21.2 mmol; 1.06 equiv.) in dichloromethane (65 ml) diisopropylethylamine (10.1 ml, 59.0 mmol) is added under stirring and cooling to 10° C. during 5 min. The obtained solution is left to heat up to the laboratory temperature while the course of the reaction is monitored with TLC. After completion of the reaction the solution is cooled down to −5° C., and a solution of (S)-3-[4-[2-(4-fluorophenyl)-[1.3]dioxan-2-yl]-1-oxobutyl]-4-phenyloxazolidin-2-one (6.62 g, 16.0 mmol) in dichloromethane (15 ml) is added under stirring during 5 min. Then, the mixture is cooled down to −32° C., and after stirring for 10 min a solution of TiCl₃(Oi-Pr) starts to be added during 20 min, which has been prepared previously by mixing of TiCl₄ (a 1M solution in CH₂Cl₂; 15.5 ml, 15.5 mmol) and Ti(Oi-Pr)₄ (1.54 g, 5.17 mmol) in CH₂Cl₂ (25 ml) at 10° C., followed by stirring for 30 min. The obtained dark solution is stirred at the same temperature for 2.5 h and then acetic acid (4.5 ml) is added during 5 min and the solution is stirred at −30° C. for another 10 min. Then, to the reaction mixture a 0.46M solution of sodium dihydrogen citrate is added (120 ml, or the other way round the reaction mixture is poured onto this buffer) as well as methylene chloride (50 ml). The reaction mixture is intensively stirred for 20 min, the organic phase is separated and the aqueous phase is extracted with CH₂Cl₂ (50 ml). The combined organic phases are washed with water (50 ml), brine (40 ml) and evaporated in a rotational vacuum evaporator. To the evaporation residue methanol (200 ml) and 10% aqueous hydrochloric acid (4.5 ml) are added and the mixture is stirred at the temperature of 25° C. overnight. The separated product is filtered off, washed with EtOH (2×10 ml) and dried. The obtained product is recrystallized from ethyl acetate (120 ml), the crystals are sucked off, washed with ethyl acetate and dried at 45° C. Melting temp. 210-213° C.

Yield: 4.69 g (51.4%) of a compound of solution IV (PG=H). HPLC: diastereoisomeric purity 98.6%.

¹H-NMR (250 MHz, CD₃SOCD₃): δ 7.95 (dd, J=8.6 Hz, J=5.7 Hz, 2H), 7.38-7.26 (m, 4H), 7.25-7.05 (m, 5H), 6.80 (t, J=8.9 Hz, 2H), 6.68 (d, J=8.5 Hz, 2H), 6.56 (m, 2H), 6.00 (d, J=9.4 Hz, 1H), 5.55 (dd, J=8.6 Hz, J=4.5 Hz, 1H), 4.76 (t, J=8.6 Hz, 1H), 4.50-4.30 (m, 2H), 4.10 (dd, J=8.7 Hz, J=4.5 Hz, 1H), 2.93 (t, J=7.3 Hz, 2H), 1.69 (m, 2H).

### EXAMPLE 6

### Preparation of the Compound of General Formula II (PG=H)

To a solution of the ketone of general formula IV (PG=H) (1.00 g, 1.59 mmol) in THF (35 ml) a 1M toluene solution of (R)-2-methyl-CBS-oxazaborolidine (0.8 ml, 0.8 mmol; 50 mol %) is added. After stirring at the laboratory temperature for 15 min a1M solution of BH₃.Me₂S in CH₂Cl₂ (3.2 ml, 2 mol equiv.) is added dropwise during 1 h and the solution is stirred at the laboratory temperature for another 30 min, the course of the reaction being monitored with TLC. The reaction is terminated by adding of MeOH (5 ml) at the temperature of 0° C. and stirring at the same temperature for 15 min. Then 1M HCl (5 ml) and water (20 ml) are added and the mixture is stirred at 0° C. for another 10 min. The organic phase is separated and the aqueous phase is extracted with dichloromethane (40 and 15 ml). The combined organic phases are washed with water (15 ml) and evaporated in a rotational vacuum evaporator. The crystalline evaporation residue is recrystallized from ethanol (10 ml). After standstill at the laboratory temperature for 1 hour and at 10° C. for 1 h the separated crystals are sucked off, washed with ethanol and dried; melting temp. 195-197° C.

Yield: 0.75 g (75%) of the compound of formula II (PG=H). HPLC: diastereoisomeric purity 98.3%.

¹H-NMR (250 MHz, CD₃SOCD₃): δ 9.29 (s, 1H), 7.30 (d, J=7.6 Hz, 2H), 7.25-7.00 (m, 9H), 6.78 (t, J=8.9 Hz, 2H), 6.66 (d, J=8.4 Hz, 2H), 6.53 (dd, J=8.9 Hz, J=4.5 Hz, 2H), 5.91 (d, J=9.2 Hz, 1H), 5.54 (dd, J=8.5 Hz, J=4.3 Hz, 1H), 5.09 (d, J=4.4 Hz, 1H), 4.74 (t, J=8.7 Hz, 1H), 4.40-4.21 (m, 3H), 4.08 (dd, J=8.8 Hz, J=4.4 Hz, 1H), 1.54-1.27 (m, 4H).

### EXAMPLE 7

### Preparation of the Compound of General Formula II (PG=Cbz)

To a suspension of the alcohol of general formula II (PG=H) (1.50 g; 2.62 mmol) in dichloromethane (30 ml) triethylamine (0.29 g, 2.88 mmol) and DMAP (12 mg) are added under stirring and cooling at 5° C. Then, a 50% toluene solution of benzyl chloroformate (1.00 ml, 2.9 mmol, 1.1 equiv.) is added dropwise during 10 min. The obtained solution is left to heat up to 10° C. during 1 h, the course of the reaction being monitored with TLC. After 2 h in total, water (25 ml) is added to the reaction mixture and the separated organic phase is washed with 1N aqueous HCl (10 ml), 9% aqueous NaHCO₃ (15 ml) and with water (15 ml) again and dried (Na₂SO₄). The crystalline evaporation residue is boiled with methanol (12 ml) and left to stand at the laboratory temperature for 1 h. The crystals are sucked off, washed with methanol and dried.

Yield: 1.69 g of a compound of formula II (PG=Cbz), i.e. 91.1%. HPLC: purity 98.3%.

### EXAMPLE 8

### Preparation of the Compound of General Formula IV (PG=Cbz)

To a suspension of the ketone of general formula IV (PG=H) (5.97 g; 9.5 mmol) in dichloromethane (100 ml) triethylamine (1.15 g, 11.39 mmol) and DMAP (15 mg) are added under stirring. Then, during cooling to 5° C., a 50% toluene solution of benzyl chloroformate (3.88 g, 11.39 mmol; 1.2 equiv.) is added dropwise in the course of 10 min. The obtained solution is left to heat up to 15° C. during 1 hour, the course of the reaction being monitored with TLC. After 2 h in total, the reaction mixture is washed with 1N aqueous HCl (30 ml), 9% aqueous NaHCO₃ (2×30 ml) and water (40 ml) again and dried (Na₂SO₄). The crystalline evaporation residue is boiled with methanol (100 ml) for 30 min and left at standstill at the laboratory temperature for 1 h. The crystals are sucked off, washed with methanol and dried. Melting temp. 178.5-179° C.

Yield: 6.45 g (96.4%) of the compound of general formula II (PG=Cbz). HPLC: purity 98.6%.

1-37. (canceled)

38. A method for the preparation of (4S)-3-{(2R,5S)-5-(4-fluorophenyl)-2-[(S)-[(4-fluorophenyl)amino](4-hydroxyphenyl)methyl]-5-hydroxypentanoyl)}-4-phenyl-1,3-oxazolidin-2-ones of general formula II 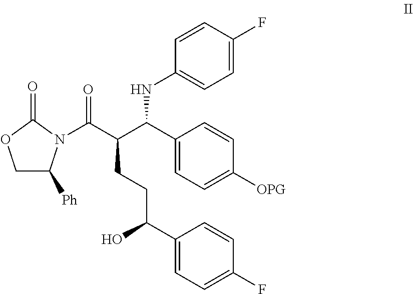
in which PG represents hydrogen or a hydroxyl protecting group selected from the group consisting of trimethylsilyl, tert-butyldimethylsilyl, benzyloxycarbonyl, tent-butoxycarbonyl, benzyl, benzhydryl or trityl,
wherein ketal oxazolidides of general formula III 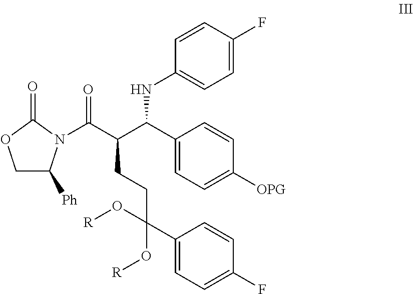
in which PG has the same meaning as above and R represents an alkyl with 1-4 carbon atoms, linear or branched, or R+R together represent a divalent alkyl, optionally substituted with 1 or 2 alkyl groups,
are deprotected by the action of acidic reagents in a mixture of water and a water-miscible solvent in the temperature range of 0 to 100° C., and the obtained ketone oxazolidide of general formula IV 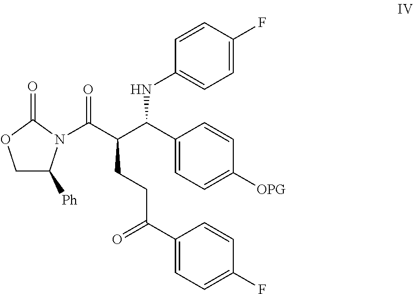
in which PG has the meaning mentioned above,
is reduced with asymmetrical reagents in an inert organic solvent in the temperature range of −30 to +40° C.

39. The method according to claim 38, wherein an organic acid selected from the group consisting of p-toluenesulfonic acid, methanesulfonic acid and acetic acid, or an inorganic acid selected from the group consisting of hydrochloric and sulfuric acid, is used as the acidic reagent for the deprotection.

40. The method according to claim 38, wherein the deprotection is carried out in a mixture of water and a water-miscible solvent selected from the group consisting of tetrahydrofuran, acetone, methyl ethyl ketone, isobutyl methyl ketone, methanol and ethanol in the temperature range of 20 to 100° C., preferably from 50° C. to the boiling temperature of the mixture.

41. The method according to claim 38, wherein a borane in the presence of a chiral ligand is used as the asymmetrical reagent for the reduction.

42. The method according to claim 38, wherein a borane complex with dimethyl sulfide, tetrahydrofuran, dimethyl aniline or diethyl aniline, is used as the borane source.

43. The method according to claim 38, wherein 2-substituted (R)-CBS-oxazaborolidine in the quantity of 5 to 20 mol %, is used as the chiral ligand.

44. The method according to claim 43, wherein 2-substituted (R)-CBS-oxazaborolidine is (R)-2-methyl-CBS-oxazaborolidine or (R)-2-(o-tolyl)-CBS-oxazaborolidine.

45. The method according to claim 38, wherein the reduction is carried out in the presence of a catalytic quantity of protic or Lewis acids selected from the group consisting of methanesulfonic acid, p-toluenesulfonic acid, trifluoroacetic acid and borotrifluoride etherate.

46. The method according to claim 38, wherein the inert organic solvent is selected from the group consisting of tetrahydrofuran, 2-methyltetrahydrofuran, tert-butyl methyl ether, toluene or dichloromethane and their mixtures.

47. The method according to claim 38, wherein the reduction is carried out at −25 to −15° C., or at 20 to +30° C.

48. The method according to claim 38, wherein hydrogen in the presence of a chiral catalyst is used as the asymmetrical reagent during the reduction.

49. The method according to claims 38, wherein gaseous hydrogen, formic acid, or its salts with amines or isopropyl alcohol are used as the source of hydrogen.

50. The method according to claim 38, wherein a transitional metal complex with iron, rhodium or ruthenium, or their combination in the presence of a chiral ligand is used as the chiral catalyst.

51. The method according to claim 38, wherein a transitional metal complex with iron, rhodium or ruthenium or their combination with a chiral ligand embedded in the molecule is used as the chiral catalyst.

52. The method according to claim 38, wherein the chiral catalyst is generated in situ.

53. The method according to claim 38, wherein (R)-4-isopropyl-2-[(R)-2-(diphenylphosphino)ferrocen-1yl]oxazoline triphenylphosphino ruthenium(II) chloride is used as the chiral catalyst.
